(19) United States
(12) Patent Application Publication
(10) Pub. No.: US 2026/0021282 A1
(43) Pub. Date: Jan. 22, 2026

(54) METHODS AND SYSTEMS FOR TRANS-CERVICAL DELIVERY OF AGENTS

(71) Applicant: Oregon Health & Science University, Portland, OR (US)

(72) Inventors: Jeffrey JENSEN, Portland, OR (US); Ov SLAYDEN, Portland, OR (US); Jian GUO, Portland, OR (US); Philberta LEUNG, Portland, OR (US)

(21) Appl. No.: 18/992,663

(22) PCT Filed: Jul. 12, 2023
(86) PCT No.: PCT/US2023/027533

§ 371 Date: Jan. 9, 2025

Related U.S. Application Data
(60) Provisional application No. 63/388,375, filed on Jul. 12, 2022.

(51) Int. Cl. A61M 25/10; A61B 17/12; A61K 9/00; A61K 31/08

(52) U.S. Cl. CPC A61M 25/1011; A61K 9/0034; A61K 31/08; A61B 17/1204; A61B 17/12136; A61M 2025/1052; A61M 2210/1425

(57) ABSTRACT

Methods and systems are provided for trans-cervical delivery of agents for various purposes, including contraception and tubal patency evaluation. The trans-cervical delivery system comprises at least one port: a tubular shaft coupled to the port, the tubular shaft including at least one hollow channel traversing an interior length of the tubular shaft; and an agent delivery end coupled to the tubular shaft, the agent delivery end including at least one flexible catheter and a plug. The trans-cervical delivery system of the present disclosure integrates a pre-formed wire frame into the agent delivery end that enables self-positioning of the catheter and assists in gaining precise access to the cornual regions of a uterus by an operator. In one example, the trans-cervical delivery system may be utilized for selective delivery of a foam agent to fallopian tubes non-surgically.

### CROSS-REFERENCE TO RELATED APPLICATION

This application claims the priority benefit of the earlier filing date of U.S. Provisional Application No. 63/388,375, filed Jul. 12, 2022, which is hereby incorporated herein by reference in its entirety.

### TECHNICAL FIELD

The present disclosure relates to the delivery of agents, such as for contraception, and more particularly to trans-cervical delivery devices and non-surgical methods for their deployment.

### BACKGROUND

A large unmet need for family planning methods, particularly permanent contraception, exists worldwide. A Trends in Contraceptive Use study conducted by the United Nations found that long-term family planning methods are the most commonly used of the contraceptive methods: 33% of women worldwide use permanent contraception and 24% use long-acting reversible methods. Among women who have completed their desired family size, permanent contraception is the most effective method of birth control; however, currently only surgical methods exist. In many low resource settings, high cost, scarcity of providers, and surgical risks limit access to this important method.

### BRIEF DESCRIPTION OF THE DRAWINGS

Embodiments will be readily understood by the following detailed description in conjunction with the accompanying drawings and the appended claims. Embodiments are illustrated by way of example and not by way of limitation in the figures of the accompanying drawings.

FIG. 1 illustrates the uterine and tubal anatomy for deployment of trans-cervical delivery devices in accordance with various embodiments;

FIG. 2 illustrates a trans-cervical delivery device according to an embodiment of the present invention;

FIG. 2A illustrates an enlarged view of an agent delivery end of a trans-cervical delivery device with a dual catheter, according to an embodiment of the present invention.

FIG. 2B illustrates various cross-sectional views of the agent delivery end of the trans-cervical delivery device with a dual catheter in accordance with FIG. 2A.

FIG. 2C illustrates a wire frame of the agent delivery end of the trans-cervical delivery device with a dual catheter in accordance with FIG. 2A.

FIG. 2D illustrates an enlarged view of a plurality of ports of the trans-cervical delivery device with a dual catheter in accordance with FIG. 2A.

FIGS. 2E and 2F illustrate various handle and port configurations in accordance with embodiments herein.

FIGS. 2G and 2H illustrate inflated plug/balloon configurations in accordance with embodiments herein.

FIGS. 21-2L are schematic representations of balloons affixed to a distal end of a catheter at various affixation points.

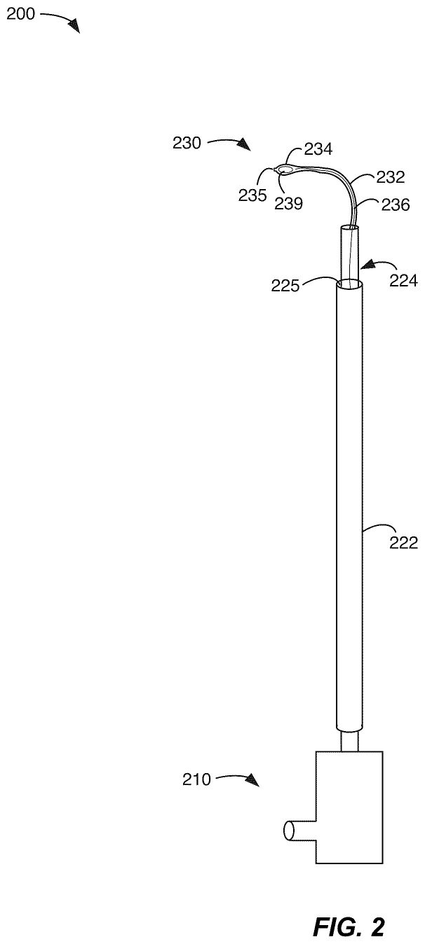

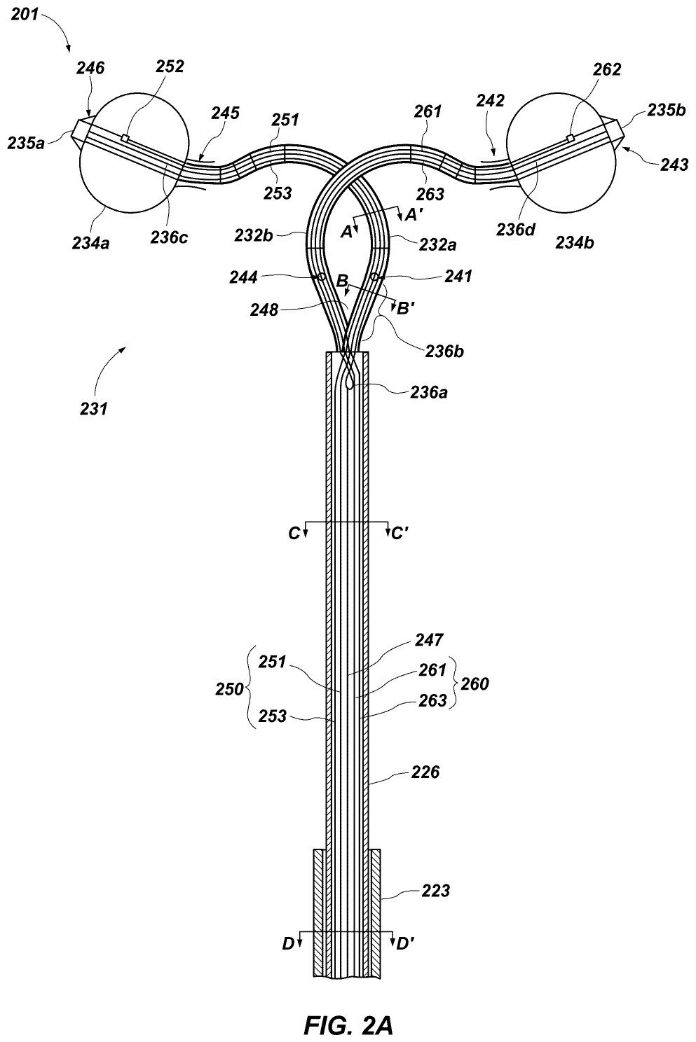

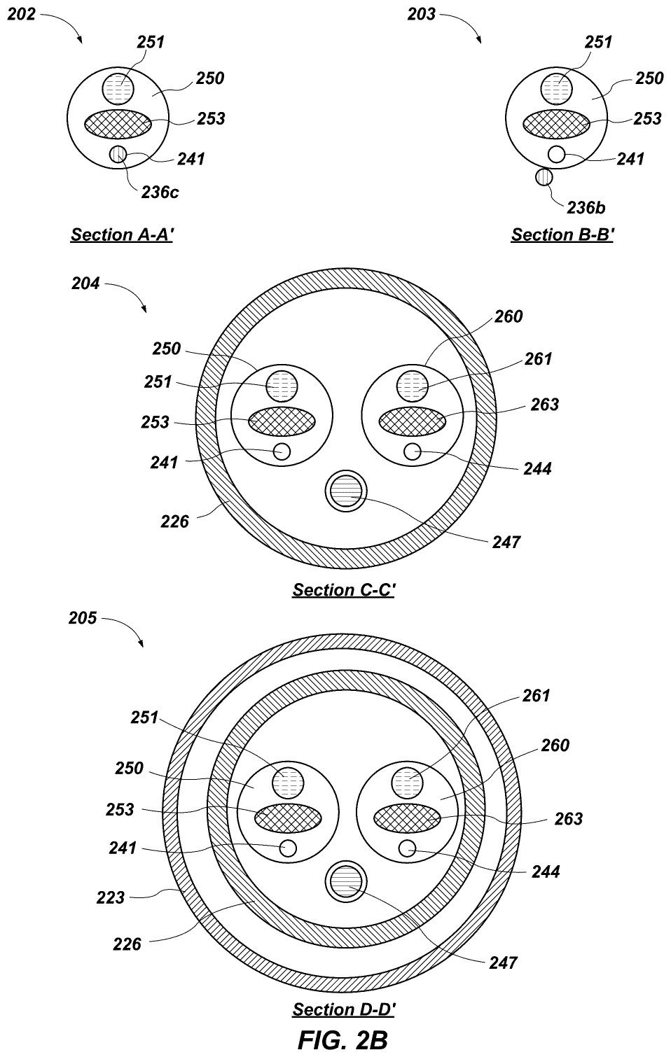

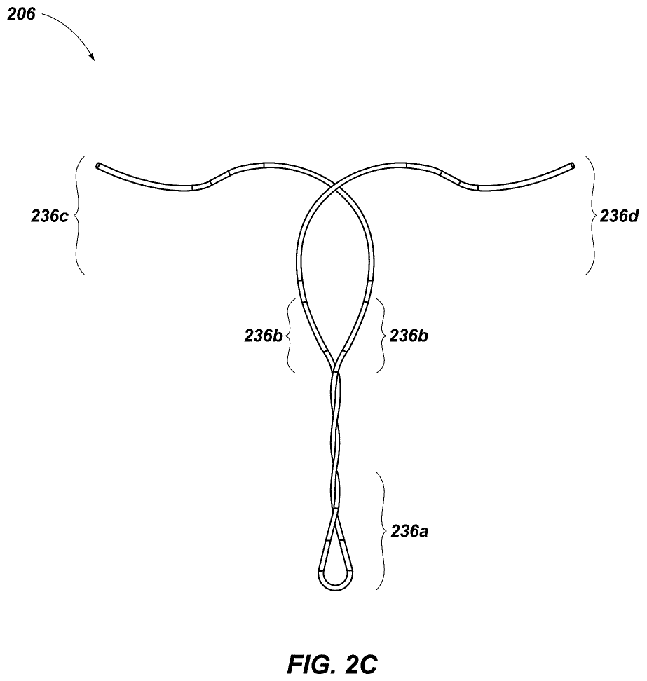

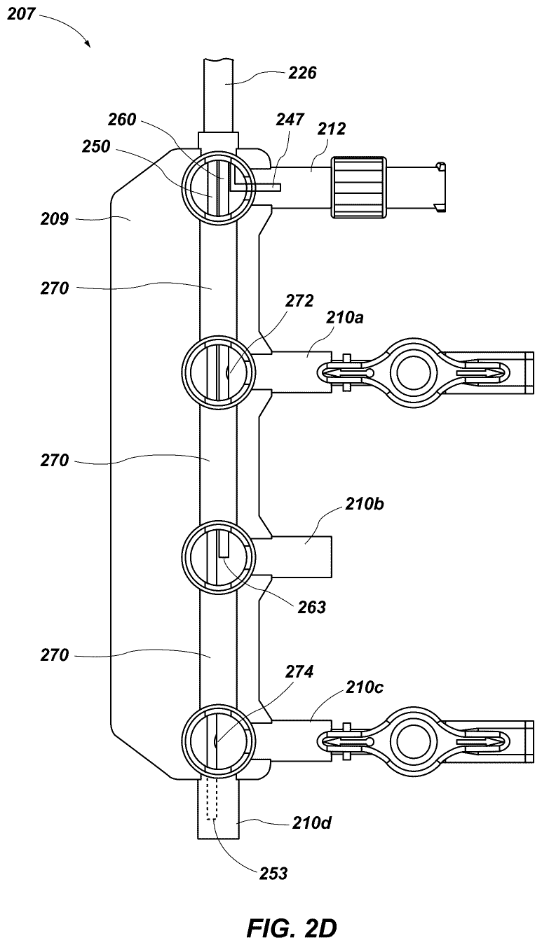

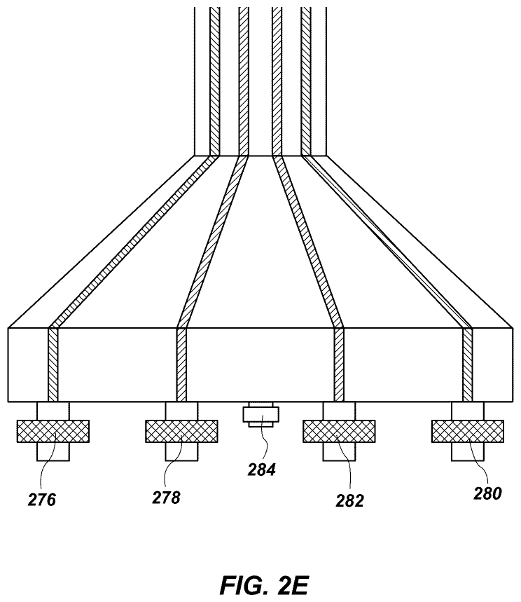

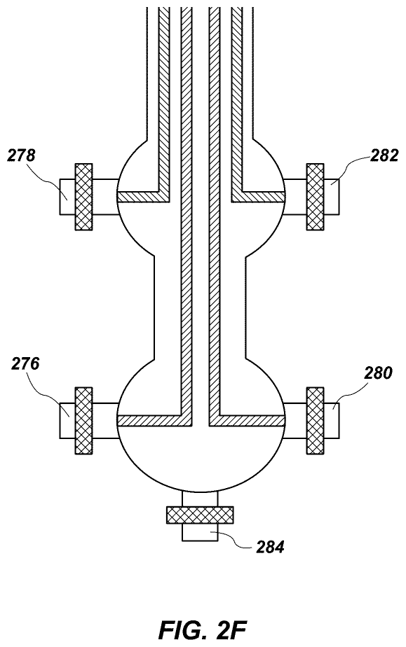

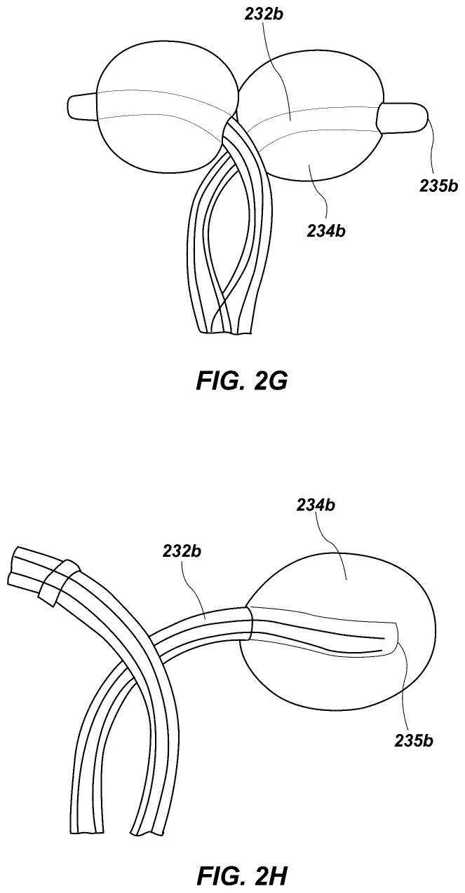

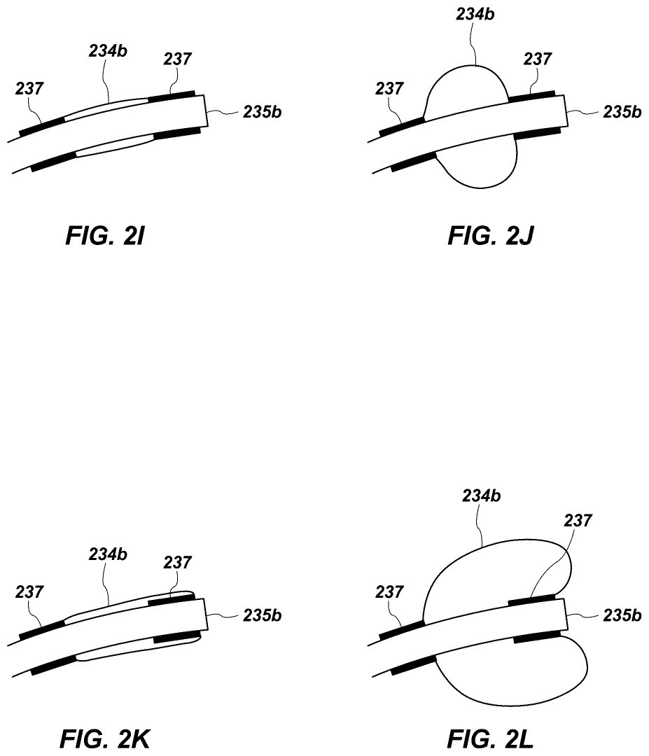

FIG. 3 illustrates a flow chart of an example method for using the trans-cervical delivery device of FIG. 2 to inhibit conception in accordance with various embodiments.

FIGS. 4A-4D are illustrations schematically showing the example method of FIG. 3 for deploying the trans-cervical delivery device in a uterus and delivering an agent in accordance with various embodiments.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order-dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A) B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

Access to the uterine cavity using a trans-cervical technique is a clinical procedure routinely performed by OB/GYN clinicians in an office setting. While some surgical trans-cervical procedures also exist (e.g., hysteroscopy), the trans-cervical technique is usually considered non-surgical, and advanced practice nurses and other healthcare workers can be trained to perform these procedures. The trans-cervical infusion or injection of a contrast medium or saline/air to the uterus has been widely utilized for hystersalpingography (HSG) and sonohysterography (SHG) to examine the uterine structure and abnormalities, tubal patency, and the effects of tubal surgery.

Current delivery devices commonly used for HSG and SHG procedures include a balloon catheter in which the inflated balloon (positioned in the lower uterine segment) provides a sole function of sealing the cervical canal after catheter insertion and prevent back flow of the contrast medium from the uterine cavity. Direct adaption of these catheter systems for trans-cervical delivery of agents (e.g., a sclerosing foam) to inhibit conception may have some limitations and clinical safety concerns, as a high injection pressure may be required to administer them into narrow fallopian tubes. The potential problems include risk of non-target exposure of agents, uneven delivery of agents into left and right fallopian tubes, pressurization of the uterine cavity, and risk of pressure-mediated vascular uptake, tissue damage, and other adverse effects.

The present disclosure is related to a delivery system and method that provide a means for administration of agent trans-cervically to the fallopian tubes independently, easily, directly, safely, and effectively in diagnostic and therapeutic procedures. The present system limits the risks associated with unwanted exposure of agent to the endometrium of the uterus, pressure-mediated tissue damage, or vascular uptake, and delivers agent trans-cervically, directly, independently and evenly to each fallopian tube with minimal loss of agent during delivery.

In accordance with an embodiment of the present disclosure, a trans-cervical delivery device is provided that integrates a pre-formed frame, such as a wire frame, into an agent delivery end of the device. The trans-cervical delivery device, according to the present disclosure, has an ability to self-position at each cornual region of a uterus without assistance of hysteroscope, fluoroscope, and other imaging equipment. The present system allows independent delivery of an agent trans-cervically to each fallopian tube with minimal exposure of agent to unintended areas such as the uterine lining. Any agent that enters the uterine cavity escapes through features/openings in the trans-cervical delivery device such that pressurization of the uterine cavity and vascular uptake does not occur. This allows safe and effective administration of treatments to the target region with the lowest possible systemic exposure.

As described herein, a trans-cervical delivery device may comprise a device for selective delivery of administered agents to the fallopian tubes without entry of any portion of the device into the fallopian tubes. An exemplary device may include two small delivery catheters supported by a nitinol wire frame skeleton introduced transcervically in a collapsed form using an insertion tube. An obturator may advance the frame and catheters past the distal end of the insertion tube to affect expansion. The nitinol memory wire frame results in a horizontal expansion of the system that automatically positions the two small balloon catheters such that one seats in each of the dual cornual regions of the uterine cavity. Inflation of the balloons isolates each uterine cornu from the rest of the uterine cavity and from each other. This allows the clinician to administer agents selectively and independently to each uterine cornu without loss of agent into the uterine cavity. If the fallopian tube is patent (open), the agent will flow into the tube. This mode of delivery increases safety when compared with existing balloon devices that administer agents directly into the uterine cavity and require pressurization of the entire endometrial cavity for flow of administered agent out the tubes. Unlike catheters designed to selectively cannulate the fallopian tube, the present device is designed such that no portion enters the fallopian tubes.

The present device can be used to assess tubal patency, impart female contraception, or to introduce an agent for imaging purposes. More specifically, aspects of the present invention include a delivery system, one or more occlusive materials, and a method for tubal occlusion and more particularly occlusion of the fallopian tubes of a female mammal for the purpose of permanent or reversible contraception.

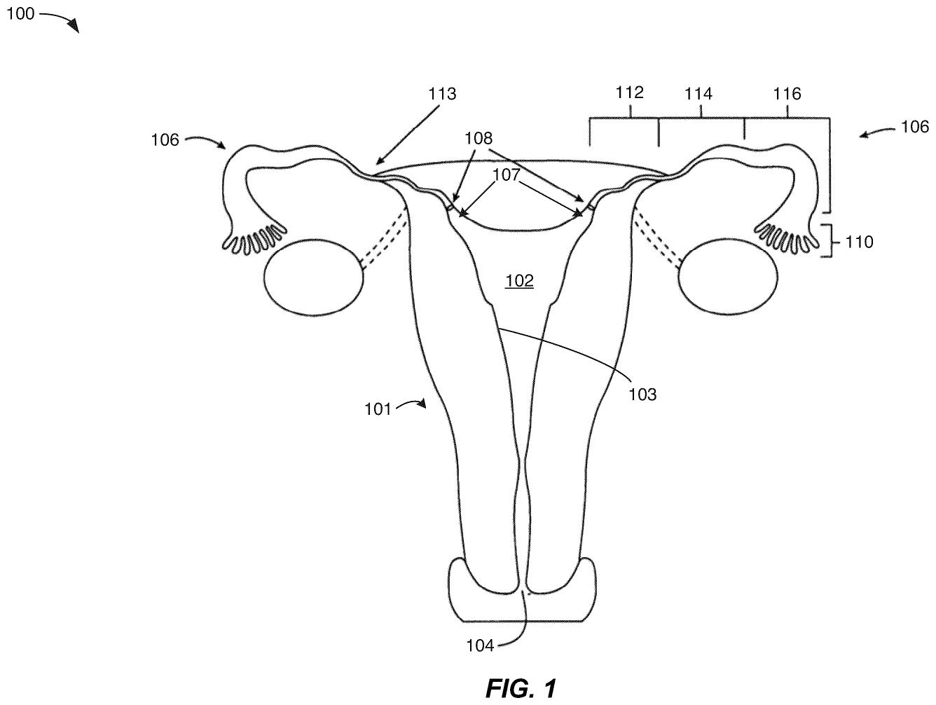

FIG. 1 depicts an anatomical view 100 of a uterus 101 and fallopian tubes 106 for deployment of trans-cervical delivery devices, according to an embodiment herein. Access to uterine cavity 102 of the uterus 101 is generally gained through cervix 104. From within uterus 101, fallopian tubes 106 are accessed via tubal ostia 108. Ostia 108 are located at cornual regions 107 of the uterus 101. Fallopian tubes 106 generally include three segments between ostia 108 and fimbria 110. Beginning adjacent cornual regions 107 of the uterus 101, an intramural segment 112 of fallopian tubes 106 are surrounded by the muscular uterine tissues. Beginning at uterotubal junction 113, fallopian tubes 106 extend beyond the uterine tissues and within the peritoneal cavity along an isthmic segment 114, and then an ampullary segment 116. It should be noted that the uterotubal junction 113 may be defined as the plane where the fallopian tube meets the peritoneal cavity.

The delivery system for trans-cervical administration of agent to the fallopian tubes 106, according to an embodiment herein, accesses the cornual regions 107 of the uterus 101 after being deployed in the uterine cavity 102. The agent is delivered to the fallopian tubes 106 in a targeted fashion by isolating the uterine cornua on each side from the main uterine cavity, in accordance with various embodiments. This minimizes contact with the endometrium 103 by the administered agent without the need for direct entry of a catheter or wire into the fallopian tube ostium. In general, the ideal placement for an agent (e.g., agents to inhibit conception) may span the intramural segment 112 to isthmic segment 114 of the fallopian tube 106.

A detailed description of the trans-cervical delivery system, according to the present disclosure, will be presented in FIG. 2. The agent in accordance with an embodiment of the present invention includes, but is not limited to, occlusive material capable of inducing tubal occlusion to prevent pregnancy, a sclerosing agent, an imaging or contrast agent, or an anti-bacterial agent.

As used herein, occlusive material comprises any natural or synthetic compositions or any combination of natural and synthetic compositions that can be placed at the desired site in the fallopian tube using the delivery systems herein. Occlusive materials may provide complete or partial occlusion that is permanent or semi-permanent, as desired for the particular application. Occlusive materials of the present disclosure may comprise materials that are foams, fluid, semi-solid, gels, and combinations thereof. The occlusive materials may further comprise a pre-formed material that is of a shape or size that occludes the fallopian tube or may be a material that will take on a form or shape or size to occlude the fallopian tube. Occlusive materials may further comprise compositions that may change physical or chemical properties in situ at the desired site in the fallopian tube. The occlusive compositions may further comprise materials that may polymerize in situ, wherein the polymerization may be initiated either at the site of interest in the fallopian tube or prior to placement at the site. Occlusive compositions may further comprise combinations of one or more of any of the foregoing materials. Disclosed herein are exemplary compositions and materials suitable for use as occlusive compositions. Furthermore, the presence or success of tubal occlusion may be verified using one or more methods, such as the further introduction of saline, foam, or radio opaque contrast agent for imaging.

Administered agents may also function as sclerosants damaging the epithelium leading to collagen replacement, but which may otherwise degrade or diffuse from the administration site and not remain within the tube or uterus permanently following treatment.

The device may also be used for the delivery of media (liquids or foam) for verification of tubal patency with or without the use of imaging (e.g. ultrasound, x-rays, or fluoroscopy)

FIG. 2 depicts a trans-cervical delivery device 200, according to the present disclosure. In the depicted embodiment, the trans-cervical delivery device 200 comprises a port 210, a tubular shaft 224, and an agent delivery end 230. As shown, the port 210 may be present on a first end of the trans-cervical delivery device 200 and the agent delivery end 230 may be present on a second, opposing end of the trans-cervical delivery device 200. The tubular shaft 224 may be configured to connect the port 210 to the agent delivery end 230, as depicted. The port 210, the tubular shaft 224, and the agent delivery end 230 of the trans-cervical delivery device 200 are coupled such that these components enclose a channel through which fluids/foams or flowable agents may pass.

The port 210 may serve as an injection port for injecting fluids/foams containing an agent (e.g., an occlusive material, sclerosant, or imaging agent) into the trans-cervical delivery device. According to the methods described in the present disclosure, during a trans-cervical administration of an agent, the port 210 of the trans-cervical delivery device 200 may remain outside of the uterine cavity for easy access.

While the illustrated example shows a single port for simplicity, it is to be understood that other embodiments may include multiple ports on the first end of the trans-cervical delivery device 200 with each port directing each input to individual channels that may carry the fluid/foam to the agent delivery end 230 of the device. For example, in one embodiment, the trans-cervical delivery device may include multiple channels or ports for balloon inflation, agent delivery, and pressure monitoring. Alternatively, a single port may be used in conjunction with a valve to control the delivery of a fluid/foam or other flowable agent into a desired channel.

The tubular shaft 224 may be a long slender structure coupling the port 210 to the agent delivery end 230. The tubular shaft 224 may also be referred to as an obturator in the present disclosure. The terms ‘tubular shaft’ and ‘obturator’ may be interchangeably used throughout the disclosure. As shown, the tubular shaft 224 includes at least one hollow channel traversing an interior length of the tubular shaft. The tubular shaft 224 may be made of standard medical-grade materials with substantial mechanical strength, including but not limited to metals or plastic such as stainless steel, nylon, PTFE, or polyurethane, FEP, pebax, HDPE, etc. In the illustrated example, the tubular shaft is made of PEEK (polyetheretherketone). According to the methods described in the present disclosure, the trans-cervical delivery device 200 is introduced into the uterine cavity using insertion tube 222. Tubular shaft 224 is used for positioning the frame to the uterine cornua.

The agent delivery end 230 of the trans-cervical delivery device 200 further comprises a flexible catheter 232 and a plug 234. A first end of the catheter 232 may run through the tubular shaft 224 and may be attached to the port 210 for control of inflation and/or foam delivery, while a second, opposing, end of the catheter 232 may be connected to the plug 234. The plug 234 may have a distal tip 235 through which an agent may be delivered to a target site. According to the methods described in the present disclosure, during a trans-cervical administration of an agent, the agent delivery end 230 of the trans-cervical delivery device 200 is deployed in the uterine cavity and the plug 234 of the agent delivery end 230 is positioned at or near the cornual regions of the uterus. The plug 234 may include an inflatable device 239, such as a balloon, which may provide a function of isolating and sealing the cornual regions to prevent leakage of the agent from the cornual regions to the uterine cavity during trans-cervical administration. While the illustrated embodiment shows a balloon as the inflatable device 239, other devices (e.g., discs, blocks, etc., whether inflatable or not and whether flexible/compressible or not) may be used in other embodiments as an alternative to the balloon, without departing from the scope of this disclosure.

As shown, a lumen of the catheter 232 of the agent delivery end 230 may include a wire frame 236. The wire frame 236 of the catheter 232 may extend to a portion of the hollow channel of the tubular shaft 224 and/or a lumen of the plug 234. The wire frame 236 may be manufactured from any material suitable for use in its intended application(s). In implementations intended for medical use, such as placement within a uterine cavity, wire frame 236 may be manufactured from a suitable biologically inert material or materials, for example, a metallic alloy. Materials may be selected for their elastic or plastic qualities, with the response to expansion and relaxation as considerations. In one embodiment, the wire frame 236 may be formed from nitinol, an intermetallic compound having approximately 50.8 atomic percent Nickel and the balance Titanium. Nitinol has the unique properties of shape memory and superelasticity. An integration of the wire frame inside the lumen of the catheter, as described in this disclosure, allows the catheter to adapt the wire pre-formed shape such that the plug and the distal tip can be freely moved, located, and positioned at the cornual region without any assistance of hysteroscope, fluoroscope, and other imaging equipment. Thus, the wire frame allows self-positioning of the agent delivery end of the device. Therefore, the wire frame not only provides flexibility to the catheter but also easy and precise access to the cornual regions by the operator.

In embodiments, the wire frame also provides a force that maintains position of the plug in the cornual area during administration of an agent. As the agent is delivered, pressure will build up in the cornua. Without the wire frame, the plug could become displaced if resistance to flow is high in the tube. However, the flexible nature of the wire allows displacement of the plug if pressure becomes excessive, as when the tube is blocked or if the plug does not position correctly. Thus, the wire frame serves several functions: 1) allows correct positioning without imaging; 2) maintains correct position during administration of an agent; and 3) increases safety by allowing displacement of the plug if delivery pressure becomes excessive.

In various embodiments, a wire may have a suitable diameter such as 0.005 inches to 0.050 inches, and have a length such as 3-10 inches.

In the illustrated example, the agent delivery end 230 of the trans-cervical delivery device 200 includes a single flexible catheter with a plug. In other embodiments, however, the agent delivery end 230 of the device may comprise a second flexible catheter (attached to the tubular shaft) with a second plug to access both the cornual regions of the uterus independently at the same time. Hence, an agent delivery end with a dual catheter and dual plugs may allow delivery of the agent to both the fallopian tubes independently. Furthermore, in an alternative embodiment, the nitinol wire frame of the catheters may be replaced with a nitinol tube for delivery of the agent. This configuration also provides the ability to inflate each inflatable device and infuse agent from separate injection ports through the catheter lumen and the plug to each fallopian tube independently.

Additionally, the trans-cervical delivery device 200 may include a movable sheath 222 surrounding a portion of the tubular shaft 224. A sliding movement of the movable sheath 222 may span the regions of the tubular shaft 224 and the agent delivery end 230. The movable sheath 222 may also be referred to as an insertion tube in the present disclosure due to its functional role in insertion of the device into a uterus. The terms ‘movable sheath’ and ‘insertion tube’ may be interchangeably used throughout the disclosure. According to the present disclosure, the movable sheath 222 is configured to be compatible with trans-cervical insertion of the device. During a trans-cervical administration of an agent, the catheter 232 with the wire frame 236 may be compressed and preloaded in the movable sheath 222. Once the trans-cervical delivery device 200 is inserted and placed in the uterine cavity, the wire frame can self-expand (e.g., bilaterally outward) after the agent delivery end is released from the movable sheath 222. In an embodiment, this operation can be accomplished by sliding the moveable sheath 222 with respect to the obturator, or advancing the obturator while maintaining the sheath in a static position.

The expansion of the wire frame allows placement of the catheter in a wide range of uterine cavity sizes and accommodate anatomic variations of the uterus. In some examples, the movable sheath 222 may also be equipped with a scale to assist the operator in positioning the movable sheath to expand the wire frame for precise placement at the cornual regions, and then to facilitate safe retraction of the wire frame after delivery of the agent for device removal. FIG. 2 depicts the trans-cervical delivery device 200 with the agent delivery end 230 of the device exposed (i.e., not inserted into the movable sheath 222). The movable sheath 222 is not only compatible with trans-cervical insertion of the device but it also functions as a drain that allows the removal of excess agent from within the uterine cavity through the cervix without causing pressure buildup, in case of back flow or leakage of agent from the cornual regions during agent delivery. This is facilitated through a gap 225 existing between the obturator and the movable sheath. As shown in FIG. 2, the gap 225 is an opening or space present between the movable sheath and the obturator within the trans-cervical delivery device 200. The gap 225 may run along an entire length of the movable sheath as well as an entire circumference of the obturator. Additionally, the movable sheath 222 may comprise a plurality of fenestrations at a distal end for drainage. Any agent that escapes or leaks into the uterine cavity during agent delivery is configured to flow out of the uterus through the gap 225 between the movable sheath and the obturator and may finally be drained through the plurality of fenestrations at the distal end of the movable sheath. In this way, the trans-cervical delivery device 200 is configured to function to deliver agents directly into the fallopian tubes without pressurizing the uterus.

Overall, the port 210, the hollow channel of the tubular shaft 224, the lumen of the catheter 232, and the inflatable device 239 of the agent delivery end 230 connect to form a channel for the injection and delivery of an agent. The trans-cervical delivery device 200 of the present disclosure may be suitable for trans-cervical administration of agents in the form of fluid, foam, gels, etc.

In an example, the trans-cervical delivery device 200 may be used for the administration of polidocanol foam into fallopian tubes to inhibit conception. Polidocanol foam is a non-hormonal sclerosant that acts locally to induce epithelial scarring and tubal occlusion for permanent contraception. The features and configurations of the trans-cervical delivery device of the present disclosure allows self-positioning of the balloon catheters and selective delivery of the polidocanol foam into fallopian tubes without any non-target effects. The detailed method of trans-cervical administration of polidocanol foam will be presented in FIGS. 4A-4D. While this example describes selective delivery of polidocanol foam to fallopian tubes, it is to be understood that other examples may include selective delivery of various other agents to fallopian tubes using the trans-cervical delivery device 200.

In various embodiments, two trilumen catheters may be connected to a handle/port and extend through an obturator to the catheter tips. One lumen is used for balloon inflation, and one for foam/fluid delivery on each side. The third lumen is used as a conduit for a frame (such as a wire frame). The frame fixes the distal catheter assembly to the obturator. Each of the catheters has a small balloon at the distal end. The handle contains several ports that connect to the various lumens of the catheters to allow independent inflation of the balloons and agent delivery on each side.

FIG. 2A provides an enlarged view of an agent delivery end 231 of a trans-cervical delivery device with a dual catheter 201, according to an embodiment of the present disclosure. The trans-cervical delivery device with a dual catheter 201 may comprise an obturator 226, which may be similar to the tubular shaft 224 of the trans-cervical delivery device 200 of FIG. 2. The trans-cervical delivery device with a dual catheter 201 may also comprise an insertion tube 223, which may be similar to the movable sheath 222 of the trans-cervical delivery device 200 of FIG. 2.

The agent delivery end 231 of the trans-cervical delivery device with a dual catheter 201 of FIG. 2A depicts a first catheter 232a and a second catheter 232b. A first end of each of the first catheter 232a and the second catheter 232b may run through the obturator 226 and may be attached to their respective ports for control of inflation of an inflatable device and/or agent delivery. A detailed description of a plurality of ports of the trans-cervical delivery device with a dual catheter 201 will be presented in FIG. 2D. A second end of the first catheter 232a may be connected to a first plug 234a. The first plug 234a may have a first distal tip 235a through which an agent may be delivered to a target site. Similarly, a second end of the second catheter 232b may be connected to a second plug 234b. The second plug 234b may have a second distal tip 235b through which an agent may be delivered to a target site. As described previously, each of the first plug 234a and the second plug 234b may include an inflatable device, such as a balloon, which may provide a function of isolating and sealing cornual regions to prevent leakage of the agent from the cornual regions to the uterine cavity during trans-cervical administration. As depicted, the inflatable device of the first plug 234a may have a first end 245 and a second end 246 which may be attached to a lumen of the first catheter 232a. Also, the inflatable device of the second plug 234b may have a first end 242 and a second end 243 which may be attached to a lumen of the second catheter 232b. During trans-cervical administration of agent, each of the first catheter 232a and the second catheter 232b is configured to independently inflate its respective inflatable device, and independently deliver an agent to a fallopian tube.

In the illustrated example of the trans-cervical delivery device with a dual catheter 201, individual channels exist for inflation of each inflatable device and delivery of agent via each catheter. For example, the first catheter 232a may comprise a first tri-lumen extrusion 250 which may include a first balloon inflation lumen 251, a first agent delivery lumen 253, and a first wire lumen 241. The first balloon inflation lumen 251 within the first tri-lumen extrusion 250 may have a notch 252 (for balloon inflation) located at the inflatable device of the first plug 234a. The second catheter 232b may comprise a second tri-lumen extrusion 260 which may include a second balloon inflation lumen 261, a second agent delivery lumen 263, and a second wire lumen 244. The second balloon inflation lumen 261 within the second tri-lumen extrusion 260 may have a notch 262 (for balloon inflation) located at the inflatable device of the second plug 234b. Each of the first tri-lumen extrusion 250 and the second tri-lumen extrusion 260 may extend from their respective catheters through the obturator 226 and may be attached to their respective ports at the plurality of ports of the trans-cervical delivery device. The plurality of ports and their details will be presented in FIG. 2D. In this way, the presence of multiple and independent channels for balloon inflation and agent delivery for each arm or catheter within the trans-cervical delivery device of the present disclosure enables delivery of agent to each fallopian tube independently.

Furthermore, a cavity pressure lumen 247 may exist within the obturator 226, in addition to the first tri-lumen extrusion and the second tri-lumen extrusion. The cavity pressure lumen 247 may extend from a region near the plurality of ports to a distal end of the obturator 226 near the agent delivery end 231. The cavity pressure lumen 247 may exist as a single lumen extrusion. A cavity pressure tube may run through the cavity pressure lumen 247 within the obturator 226. As shown, a free end 248 of the cavity pressure tube may be projected outside of the obturator 226. When the device is deployed, the free end 248 of the cavity pressure tube may be situated in the uterine cavity and may be used to monitor pressure in the uterus, e.g., via a pressure transducer attached to a proximal end of the cavity pressure lumen 247 near the plurality of ports. This provides a safety feature to ensure that the uterus is not being pressurized during agent delivery.

In an embodiment, a pressure monitoring system may include a catheter filled with sterile water or saline positioned at the distal end of the obturator and connected via a Luer lock to a single use sterile device. The device may have a sealed pressure sensor that is compatible with inline fluid delivery through a Luer lock connector. The output cord may connect to an external pressure computer system for monitoring and recording of pressure. In some embodiments, the output may also connect to one or more mechanical pressure gauges.

As described previously with reference to FIG. 2, the trans-cervical delivery device with a dual catheter 201 of FIG. 2A also comprises a nitinol wire frame that is configured to provide flexibility to the agent delivery end 231 of the device. As depicted in FIG. 2A, the nitinol wire frame may be a single piece of a wire that is twisted to form a proximal end 236a, a first region 236b, a second region 236c, and a third region 236d. The proximal end 236a of the wire frame may be configured to remain outside of the first and second tri-lumen extrusions, but may be configured to remain inside of the obturator 226. The proximal end 236a of the wire frame may be fixed in place inside the obturator 226. The first region 236b of the wire frame is also configured to remain outside of the first and second tri-lumen extrusions. The second region 236c of the wire frame is inserted into the first wire lumen 241 of the first tri-lumen extrusion 250 within the first catheter 232a via an opening, as depicted. The third region 236d of the wire frame is inserted into the second wire lumen 244 of the second tri-lumen extrusion 260 within the second catheter 232b via an opening, as shown. Thus, the second region 236c and the third region 236d of the wire frame run inside their respective tri-lumen extrusions and may extend up to the first distal tip 235a and the second distal tip 235b of the agent delivery end 231, respectively. The dual catheter configuration of the trans-cervical delivery device, as illustrated in FIG. 2A, allows self-positioning of the agent delivery end of the device due to wire geometry and material (nitinol) properties. As such, an important advantage is that the device can be used for trans-cervical procedures without concurrent imaging technologies.

FIG. 2B shows various cross-sectional views of the obturator 226 and one of the catheters of the agent delivery end 231 of the trans-cervical delivery device with a dual catheter 201 in accordance with FIG. 2A, depicting internal structures. For example, a first cross-sectional view 202 represents section A-A′ of the first catheter 232a, a second cross-sectional view 203 represents section B-B′ of the first catheter 232a, a third cross-sectional view 204 represents section C-C′ of the obturator 226 without the insertion tube 223, and a fourth cross-sectional view 205 represents section D-D′ of the obturator 226 with the insertion tube 223.

The first cross-sectional view 202 shows an interior of the first tri-lumen extrusion 250, indicating the first balloon inflation lumen 251, the first agent delivery lumen 253, and the first wire lumen 241. The first cross-sectional view 202 also includes the second region 236c of the wire frame inside the first wire lumen 241. The second cross-sectional view 203 shows an interior of a portion of the first tri-lumen extrusion 250 which does not include a wire frame. As such, the second cross-sectional view 203 indicates the first balloon inflation lumen 251, the first agent delivery lumen 253, and an empty first wire lumen 241. Since the first region 236b of the wire frame exists outside of the tri-lumen extrusion, the second cross-sectional view 203 appreciates this location of the first region 236b of the wire frame being shown outside of the cross-section.

The third cross-sectional view 204 shows an interior of the obturator 226, showing the first tri-lumen extrusion 250, the second tri-lumen extrusion 260, and the cavity pressure lumen 247. The first tri-lumen extrusion 250 further includes the first balloon inflation lumen 251, the first agent delivery lumen 253, and an empty first wire lumen 241. The second tri-lumen extrusion 260 further includes the second balloon inflation lumen 261, the second agent delivery lumen 263, and an empty second wire lumen 244. Both the first wire lumen 241 and the second wire lumen 244 are empty and sealed as the nitinol wire frame does not extend through the obturator 226 at this level.

The fourth cross-sectional view 205 shows an interior of a region of the obturator 226 which is enclosed by the insertion tube 223. The interior of the obturator 226 shown in the fourth cross-sectional view 205 is the same as the interior of the obturator 226 shown in the third cross-sectional view 204.

The first balloon inflation lumen 251 of the first tri-lumen extrusion 250 may carry air or fluid (such as saline) to inflate the inflatable device of the first plug 234a. The second balloon inflation lumen 261 of the second tri-lumen extrusion 260 may carry air or fluid to inflate the inflatable device of the second plug 234b. The first agent delivery lumen 253 of the first tri-lumen extrusion 250 may carry an agent to be delivered to a fallopian tube via the first catheter 232a. The second agent delivery lumen 263 of the second tri-lumen extrusion 260 may carry an agent to be delivered to a fallopian tube via the second catheter 232b. The cavity pressure lumen 247 within the obturator 226 serves as a catheter to monitor pressure in the uterus, as described previously.

FIG. 2C illustrates an enlarged view 206 of the wire frame of the agent delivery end 231 of the trans-cervical delivery device with a dual catheter 201 of FIG. 2A. As described previously, the wire frame may be made of a metallic alloy, such as nitinol, with elastic or plastic qualities that shows response to expansion and relaxation. As shown in FIG. 2C, the wire frame may be a single piece of a wire that may be twisted to form a proximal end 236a, a first region 236b, a second region 236c, and a third region 236d. As depicted, the second region 236c and the third region 236d of the wire frame may form two arm-like structures of the nitinol wire. The proximal end 236a of the wire frame may include the twisted configuration and may be attached to an inside of the obturator, but located outside of the tri-lumen extrusions. The first region 236b of the wire frame may be configured to be located outside of the tri-lumen extrusions. As described previously, the second region 236c and the third region 236d of the wire frame may be inserted into the tri-lumen extrusions of the first catheter 232a and the second catheter 232b of the device, respectively. The interaction of the two arm-like structures of the nitinol wire enables self-positioning of the delivery catheters and the balloons to the cornual regions through a spring-like action, as the device is advanced to the fundus of the uterus.

In embodiments, the wire frame may be constructed from more than one piece of wire, such as with two pieces of pre-formed and shaped wires bound or glued together.

While the description above describes the frame with respect to a metallic material, such as nitinol, it should be appreciated that other metals may be used, such as stainless steel or titanium, and other non-metallic materials may be used, such as plastics or shape-memory polymers.

FIG. 2D illustrates an enlarged view 207 of a plurality of ports of the trans-cervical delivery device with a dual catheter of FIG. 2A. As shown, the region of the plurality of ports may optionally include a handle 209. The handle 209 may be connected to a proximal end of the obturator 226. The handle 209 may comprise four different ports, a first port 210a, a second port 210b, a third port 210c, and a fourth port 210d. As depicted, the first tri-lumen extrusion 250 and the second tri-lumen extrusion 260 may run through the handle 209 and communicate with these ports. For example, a proximal end of the first agent delivery lumen 253 of the first tri-lumen extrusion 250 communicates with the fourth port 210d, such that an agent may be inserted through the fourth port 210d to be delivered via the first catheter 232a. A proximal end of the second agent delivery lumen 263 of the second tri-lumen extrusion 260 communicates with the second port 210b, such that an agent may be inserted through the second port 210b to be delivered via the second catheter 232b. The first balloon inflation lumen 251 of the first tri-lumen extrusion 250 communicates with the third port 210c via a skive 274 such that the inflatable device of the first plug 234a may be inflated by injecting air or fluid through the third port 210c. The second balloon inflation lumen 261 of the second tri-lumen extrusion 260 communicates with the first port 210a via a skive 272 such that the inflatable device of the second plug 234b may be inflated by injecting air or fluid through the first port 210a. A proximal end of each of the first balloon inflation lumen 251 and the second balloon inflation lumen 261 is sealed, such that they do not communicate with the fourth port 210d and the second port 210b, respectively. Additionally, a proximal end of the first wire lumen 241 of the first tri-lumen extrusion 250 and a proximal end of the second wire lumen 244 of the second tri-lumen extrusion 260 are also sealed, such that they do not communicate with the fourth port 210d and the second port 210b, respectively. In the illustrated example, the second tri-lumen extrusion 260 may be shorter than the first tri-lumen extrusion 250 (e.g., by an inch or so) for communication through the first port 210a and the second port 210b.

The cavity pressure lumen 247 may communicate with a fifth port 212 and pressure may be monitored in the uterus by connecting the fifth port 212 to a pressure transducer, for example. A filler adhesive 270 may be introduced into channels between ports to isolate them. Thus, each port communicates independently through its respective lumen to its output at distal end of device.

FIG. 2D illustrates one configuration of the handle/ports, although it should be appreciated that a large number of alternative arrangements may be used, without departing from the present disclosure. FIGS. 2E and 2F provide two such alternatives.

FIG. 2E illustrates a handle/port configuration in which the ports are located at the proximal end of the handle/device. This may provide a more consistent region of access for clinicians or health workers to inflate the balloons and infuse agents. FIG. 2E shows left infusion port 276, left inflation port 278, right infusion port 280, right inflation port 282, and pressure monitor/port 284.

FIG. 2F illustrates a further alternative in which the ports are separated on opposing sides of the handle. FIG. 2F shows left infusion port 276, left inflation port 278, right infusion port 280, right inflation port 282, and pressure monitor/port 284. An advantage of this configuration is reduction of the risk of confusion by operators during use.

In various embodiments, a balloon may be provided at the distal tip of the catheter such that the balloon invaginates the catheter tip to prevent the catheter tip from embedding or pressing into the uterine wall. In addition, such an embodiment allows a small space to develop at the outflow end of the distal tip which allows the agent to pressurize prior to passing into the small tubal ostium. This facilitates delivery of the administered agent into the fallopian tube without the need for exact positioning of the catheter tip into the fallopian tube ostia.

While various configurations of catheter/balloon affixation are contemplated herein, two different embodiments are illustrated. FIG. 2G shows a catheter 232b with a plug/balloon 234b where, when the plug/balloon 234b is inflated, the distal/infusion tip 235b extends past the plug/balloon 234b. In contrast, FIG. 2H shows a catheter 232b with a plug/balloon 234b where, when the balloon 234b is inflated, the distal/infusion tip 235b is invaginated or encompassed by the balloon 234b. The configuration of FIG. 2H is accomplished by affixing the plug/balloon closer to the distal tip of the catheter and/or by inverting the plug/balloon before affixing it at the distal tip of the catheter.

As an example, the balloon may be affixed to the catheter, and inflated through a small skive created in the catheter to access the balloon inflation lumen. In an embodiment, the balloon may be a small length of silicone tube that is glued to the catheter. The balloon may be glued proximal and distal, toward the distal end of the catheter. This configuration allows the balloon to expand and invaginate the distal tip of the catheter.

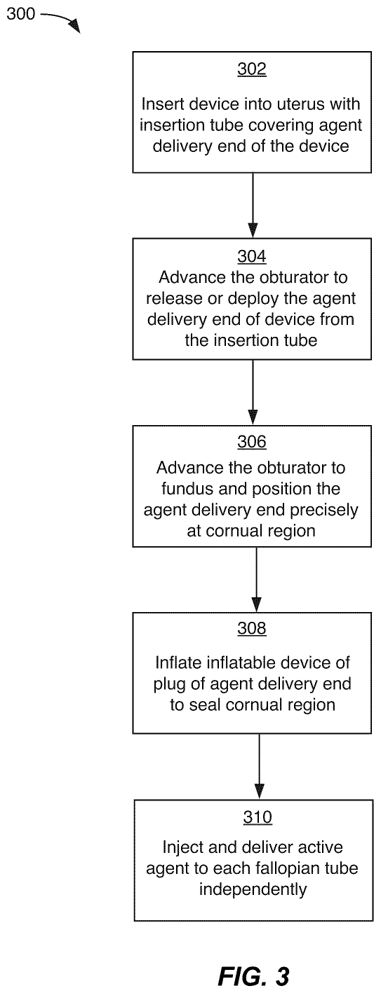

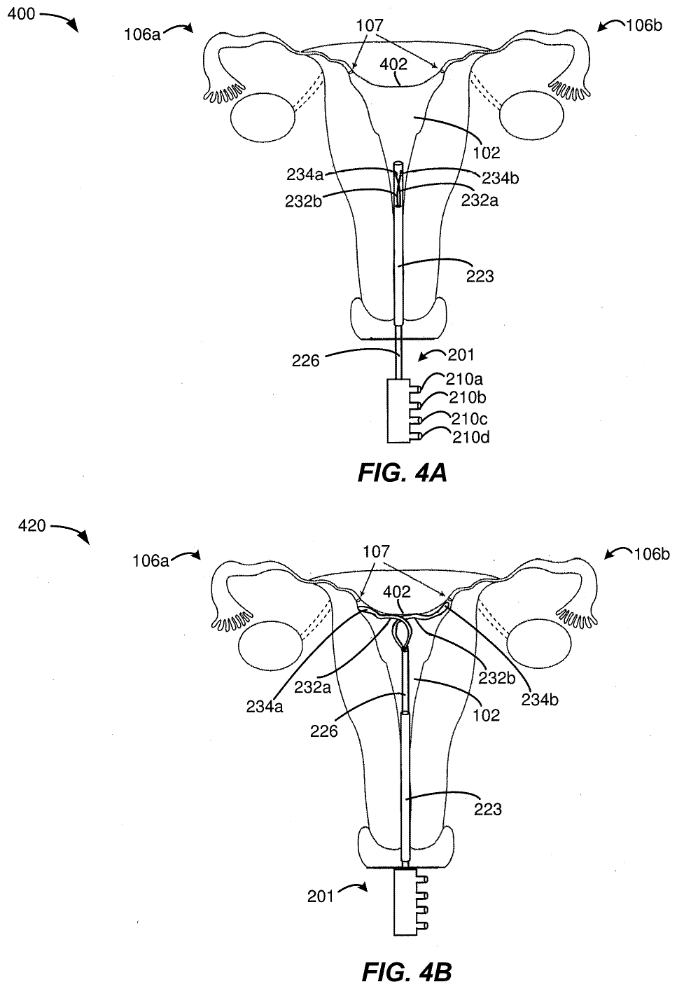

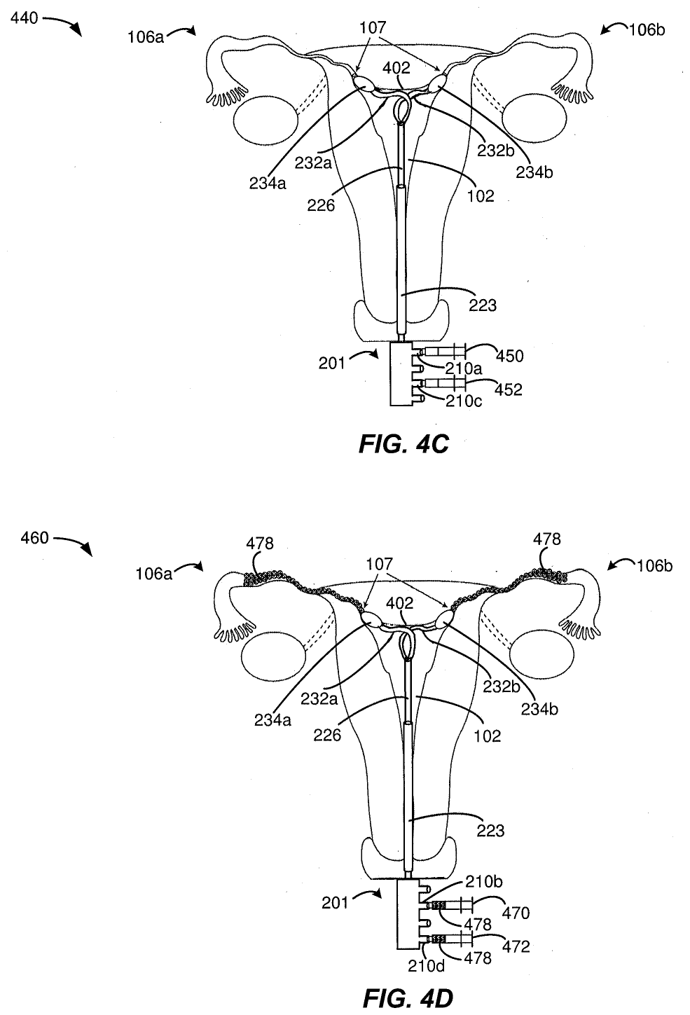

FIGS. 21-2L provide schematic representations of balloons 234b affixed to a distal end 235b of a catheter at affixation points 237. In particular, FIGS. 2K and 2L show the inversion of balloon 23b at the distal fixation locations, which, upon inflation, allows the balloon 234b to extend beyond the distal tip 235b.

FIG. 3 provides the operations of an example method 300 for using a trans-cervical delivery device, such as the trans-cervical delivery device 200 of FIG. 2 or the trans-cervical delivery device with a dual catheter 201 in accordance with FIG. 2A, for delivering an agent to each fallopian tube independently to inhibit conception. The operations of method 300 may be carried out in whole or in part using the trans-cervical delivery device.

In operation 302, the trans-cervical delivery device is inserted into lower uterine segment. During the insertion, an agent delivery end of the trans-cervical delivery device is preloaded in a movable sheath or insertion tube such that the insertion tube covers the agent delivery end of the device. The placement is guided by the length of the uterine cavity. This distance is determined by the clinician by using a uterine sound, a device specifically designed for this purpose. The insertion tube is positioned in the lower uterine segment. Typically, this is 2-3 cm below the fundus (sounded distance).

Following placement of the insertion tube, in operation 304, an obturator of the device is advanced approximately 1 cm within the uterine cavity to release the agent delivery end from the insertion tube. Thus, the advancement of the obturator deploys the agent delivery end, wherein a wire frame of a catheter of the agent delivery end self-expands for placement within the lower uterine segment.

In operation 306, the obturator of the trans-cervical delivery device is further advanced to the fundus to the full distance as determined by sounding such that the agent delivery end reaches a distant position within the uterine cavity. The shape memory and flexible properties of the wire frame allow self-positioning of the catheter such that a plug of the agent delivery end is positioned precisely at the cornual region of the uterus.

In operation 308, an inflatable device within the plug of the agent delivery end is inflated by injecting air or fluid. The inflation of the inflatable device isolates and seals the cornual region of the uterus, thereby preventing leakage of agent from the cornual region to the uterine cavity during trans-cervical administration. Finally, in operation 310, the agent is injected into the trans-cervical delivery device via a port of the trans-cervical delivery device, the port being disposed outside of the uterine cavity. The injected agent travels through the port, the obturator, and the catheter of the trans-cervical delivery device and is selectively delivered to a fallopian tube via a distal tip of the plug of the agent delivery end of the trans-cervical delivery device.

In embodiments where the agent delivery end of the trans-cervical delivery device includes two wire framed catheters with each catheter having an individual plug, it may allow trans-cervical administration of agent to each fallopian tube independently and/or sequentially. This feature of having individual catheters or arms for each fallopian tube makes the trans-cervical delivery device of the present disclosure more advantageous over other non-selective delivery devices, as the clinician can individually evaluate and treat each side independently. The dual wire also provides additional stability due to the spring like action of the wire frame. Alternatively, a delivery device may be configured with a single side or arm such that delivery may be made to one fallopian tube, or serially to both fallopian tubes.

It will be appreciated that the trans-cervical delivery device, in the disclosed embodiments, may operate in a range of uterine cavity sizes and accommodate anatomic variations of the uterus (e.g., normal uterine sound length of 6-10 cm). Other sizes could be formed, as needed, to accommodate different anatomies. The self-expansion and shape memory of wire frames of the catheters allow precise placement and accurate positioning of the catheters within the uterine cavity. In typical use, when wire frames of the catheters are expanded from their compressed state after the agent delivery end is released from the insertion tube, the catheters may first come into contact with the fundus of the uterus, but the wire frame assists in directing the tips to the cornual region. The fundus may further direct the wire frame and the catheters to be expanded and molded such that the individual plugs of the agent delivery end are correctly positioned at the cornual regions. The spring action of the wire permits lateral expansion for a larger uterine cavity size, and medial collapse for a smaller cavity. The trans-cervical delivery device, in the disclosed embodiments, can independently and effectively deliver an agent to fallopian tubes of different diameters or resistance, assisted by the spring action of the frame.

In FIGS. 4A-4D, the application of the trans-cervical delivery device in the context of a uterus and fallopian tubes is schematically depicted in accordance with the present disclosure. The uterus and fallopian tubes depicted in FIGS. 4A-4D may be similar to the uterus 101 and fallopian tubes 106 of FIG. 1. The trans-cervical delivery device shown in FIGS. 4A-4D may be similar to the trans-cervical delivery device with a dual catheter 201 of FIG. 2A. As such, components previously introduced are numbered similarly in these figures and are not re-introduced for brevity. FIGS. 4A-4D will be described herein collectively.

As described herein, the present device is designed not to enter the fallopian tubes. Instead, the device isolates/seals the tubal cornua, and this permits delivery of agent into the fallopian tubes without the need for the catheter tip to enter the tubal ostia. In an embodiment, this is caused by pushing the agent into a space that develops, between the balloon and the portion of the cavity within the cornual region that includes the ostia of the fallopian tubes, during administration. In embodiments, an agent could be delivered to the cornua directly, but the device would not enter the fallopian tubes.

In FIG. 4A, a first anatomical view 400 shows insertion of the trans-cervical delivery device with a dual catheter 201 into a lower segment of the uterine cavity 102. The trans-cervical delivery device with a dual catheter 201 may comprise four ports, a first port 210a, a second port 210b, a third port 210c, and a fourth port 210d, all of them disposed outside of the uterine cavity 102. Additionally, the agent delivery end of the trans-cervical delivery device with a dual catheter 201, in the illustrated example, includes a first catheter 232a with a first plug 234a and a second catheter 232b with a second plug 234b. A wire lumen of a tri-lumen extrusion of each of the first catheter 232a and the second catheter 232b is integrated with flexible wire frames, as described previously with reference to FIG. 2A. As shown, each of the first catheter 232a and the second catheter 232b is compressed and preloaded in a movable sheath or an insertion tube 223 of the trans-cervical delivery device 201, the insertion tube 223 being compatible with trans-cervical insertion. Thus, the insertion tube 223 surrounds or covers the dual catheters (with the plugs) of the agent delivery end of the inserted trans-cervical delivery device 201, in FIG. 4A.

As shown in a second anatomical view 420 in FIG. 4B, an advancement of a tubular shaft or an obturator 226 of the device into the uterine cavity uncovers the agent delivery end of the inserted trans-cervical delivery device 201, thereby exposing the first catheter 232a and the second catheter 232b within the uterine cavity 102. A brief delay allows the catheters to deploy. The wire frames of each of the first catheter 232a and the second catheter 232b began to self-expand soon after the advancement of the obturator 226, and the flexible properties of the wire frames allow deployment and positioning of the catheters within the uterine cavity 102. As the obturator 226 of the inserted trans-cervical delivery device 200 is further advanced to a distant position within the uterine cavity 102, the catheters may come in contact with the fundus 402 of the uterus. The fundus 402, in turn, reorients the catheters to be molded (due to the spring action of the frame) such that the first plug 234a and the second plug 234b are precisely positioned at the cornual regions 107 of the uterus, as depicted in FIG. 4B. The shape memory of the wire frames of the catheters helps retain the plugs at the cornual regions.

As depicted in a third anatomical view 440 in FIG. 4C, an inflatable device within the first plug 234a of the agent delivery end is inflated by injecting air or fluid via the third port 210c using a syringe 452. An inflatable device within the second plug 234b of the agent delivery end is inflated by injecting air or fluid via the first port 210a using a syringe 450. The inflation of the inflatable devices within the plugs, as depicted in FIG. 4C, isolates and seals the cornual regions 107 of the uterus, thereby preventing leakage of agent from cornual regions to the uterine cavity during trans-cervical administration.

In FIG. 4D, trans-cervical administration of an agent is depicted in a fourth anatomical view 460. In the illustrated example, the agent used is polidocanol foam 478, a non-hormonal sclerosant agent which inhibits conception. As shown, the foam 478 is injected into the inserted trans-cervical delivery device 201 via the second port 210b of the trans-cervical delivery device 201 using a syringe 470. The foam 478 injected into the second port 210b travels through a second tri-lumen extrusion within the obturator 226 and then through the second catheter 232b and is independently delivered to a second fallopian tube 106b via a distal tip 235b of the second plug 234b of the agent delivery end of the trans-cervical delivery device 201. As depicted in FIG. 4D, the foam 478 is also injected via the fourth port 210d of the trans-cervical delivery device 201 using a syringe 472. The foam 478 injected into the fourth port 210d travels through a first tri-lumen extrusion within the obturator 226 and then through the first catheter 232a and is independently delivered to a first fallopian tube 106a via a distal tip 235a of the first plug 234a of the agent delivery end of the trans-cervical delivery device 201.

After delivering the agent to the target site, the inflatable device within each of the first plug 234a and the second plug 234b of the agent delivery end is deflated and the trans-cervical delivery device 200 is removed from the uterine cavity 102 by pulling the obturator 226 back into the insertion tube 223. While the illustrated example method utilizes a trans-cervical delivery device with dual catheters and dual ports, other examples may use trans-cervical delivery devices with variations in number of ports and/or number of catheters, without departing from the scope of this disclosure.

In this way, trans-cervical administration of an agent may be carried out safely and effectively in a non-surgical fashion. The present system reduces cost and complexity associated with the trans-cervical procedures, and increase access particularly in low resource settings. A technical effect of the trans-cervical delivery system and method, according to the present disclosure, is that it causes occlusion or blockage of the fallopian tubes without leaving any medical device in place. During administration, the self-positioning of the agent delivery end of the system at the uterine cornua and selective delivery of the agent to the fallopian tubes without pressurization of the uterine cavity minimizes non-target effects or exposure of agent to the uterine lining. The present system allows delivery of a small dose of agent, thereby causing minimal waste of agent delivered trans-cervically. In addition, the present system allows excellent conformance to anatomic variations of uterus and the system has the ability to position correctly in a broad range of normal uterine sizes. In the illustrated example, the system is utilized for delivering an agent to fallopian tubes to inhibit conception.

Furthermore, the systems and methods disclosed herein may be useful for several additional purposes beyond female permanent contraception. For example, the present system may also be used to deliver fluid compositions comprising therapeutic agents for treatment of one or more fallopian tubes, in other embodiments. In one example, the fluid composition providing therapeutic treatment may be provided to the interior of the uterine cornua, to one or more areas of the interior lumen of a fallopian tube, to the fimbrae, to an ovary surface, or to the peritoneum. In some examples, the composition may include, but may not be limited to, hormones, fertility enhancing compounds, fertility interfering compounds, motility enhancing compounds, motility interfering compounds, compounds affecting the cilia/deciliation cycle, cilia growth enhancing or interfering compounds, ovarian follicle treatment compounds, endometriosis treatment compounds, antibacterial, antimicrobial, antifungal, antiviral, antimycoplasmal, or antiparisital compounds, compounds that reduce inflammation or scar tissue formation, and others that may treat or prevent conditions related to the fallopian tube, uterus, ovaries, or other organs or coverings reached by a composition flowing from the cornua or ostia of a fallopian tube.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

The following claims particularly point out certain combinations and sub-combinations regarded as novel and nonobvious. These claims may refer to “an” element or “a first” element or the equivalent thereof. Such claims should be understood to include incorporation of one or more such elements, neither requiring nor excluding two or more such elements. Other combinations and sub-combinations of the disclosed features, functions, elements, and/or properties may be claimed through amendment of the present claims or through presentation of new claims in this or a related application. Such claims, whether broader, narrower, equal, or different in scope to the original claims, also are regarded as included within the subject matter of the present disclosure.

What is claimed is:

1. A trans-cervical delivery system, comprising:
at least one port;
a tubular shaft coupled to the port, the tubular shaft including at least one hollow channel traversing an interior length of the tubular shaft; and
an agent delivery end coupled to the tubular shaft, the agent delivery end including at least one flexible catheter with a plug.

2. The system of claim 1, further comprising a movable sheath surrounding the tubular shaft and the agent delivery end.

3. The system of claim 2, wherein an advancement of the tubular shaft releases the agent delivery end from the movable sheath.

4. The system of claim 1, wherein the plug includes an inflatable balloon located at a distal tip of the flexible catheter.

5. The system of claim 1, wherein the inflatable balloon is inverted at its distal end and affixed to the flexible catheter such that, after inflation, the inflated balloon invaginates the distal tip of the flexible catheter.

6. The system of claim 1, wherein a lumen of the flexible catheter includes a wire or plastic frame.

7. The system of claim 6, wherein the frame is composed of a flexible alloy material.

8. The system of claim 1, wherein the agent delivery end includes a second flexible catheter with a second plug, and wherein the second plug includes a second inflatable balloon located at a distal tip of the second flexible catheter.

9. The system of claim 1, wherein a fluid or foam agent is injected through the port and directly administered to a fallopian tube via the agent delivery end.

10. A method of delivering an agent into a female body, comprising:
inserting a delivery device into a uterine cavity with a movable sheath covering an agent delivery end of the delivery device;
uncovering the agent delivery end of the inserted delivery device by advancing a tubular shaft of the inserted delivery device;
positioning the uncovered agent delivery end of the inserted delivery device such that a plug of the agent delivery end is placed at a cornual region of the uterine cavity, wherein the plug comprises an inflatable balloon;
inflating the inflatable balloon to isolate and seal the cornual region; and
injecting an agent into the inserted delivery device and delivering the agent to a fallopian tube via the agent delivery end.

11. The method of claim 10, wherein the advancement of the tubular shaft allows a wire frame of a catheter of the agent delivery end to self-expand for placement of the agent delivery end to the cornual region within the uterine cavity.

12. The method of claim 11, wherein inflating the inflatable balloon includes injecting air or fluid through a first port of the delivery device, the port being disposed outside of the uterine cavity.

13. The method of claim 12, wherein the agent is injected into the delivery device via a second port of the delivery device.

14. The method of claim 13, wherein the injected agent travels through the second port and through a hollow channel of a tubular shaft coupling the port to the agent delivery end, and wherein the injected agent is selectively delivered to the fallopian tube via a lumen of the catheter and the agent delivery end of the delivery device.

15. The method of claim 14, wherein the agent is a polidocanol foam which inhibits conception.

16. The method of claim 11, wherein the wire frame includes a spring action to assist in seating the delivery end in the cornual region and maintaining positioning as pressure develops during delivery of an agent.

17. The method of claim 10, further comprising deflating the inflatable balloon and removing the delivery device from the uterine cavity after delivering the agent.

18. A device for trans-cervical delivery of agents, comprising:
a handle including a plurality of ports;
an obturator coupled to the handle, the obturator including a first tri-lumen extrusion forming a first flexible catheter and a second tri-lumen extrusion forming a second flexible catheter, wherein the first and second flexible catheters are in fluid communication with the plurality of ports;
a movable insertion tube surrounding the obturator; and
an agent delivery end including a distal end of the first flexible catheter and a distal end of the second flexible catheter;
wherein the agent delivery end is configured to self-position within a uterus, and wherein each of the first flexible catheter and the second flexible catheter of the agent delivery end is configured to deliver agents independently to a fallopian tube.

19. The device of claim 18, wherein a drainage space exists between the insertion tube and the obturator.

20. The device of claim 18, wherein each of the first tri-lumen extrusion and the second tri-lumen extrusion includes an agent delivery lumen, a balloon inflation lumen, and a frame lumen.

21. The device of claim 20, wherein the frame lumen of each of the first flexible catheter and the second flexible catheter includes a wire frame, and wherein the wire frame is composed of a flexible alloy material.

22. The device of claim 18, wherein each of the first flexible catheter and the second flexible catheter includes an inflatable balloon attached to a respective distal tip of each catheter.

23. The device of claim 18, wherein the obturator further comprises a cavity pressure lumen, and wherein the cavity pressure lumen carries a cavity pressure tube to monitor pressure in the uterus.
